(19) United States
(12) Patent Application Publication
(10) Pub. No.: US 2006/0182778 A1
(43) Pub. Date: Aug. 17, 2006

(54) Suture and graft delivery systems

(76) Inventors: Nilesh Balar, Palisades Park, NJ (US); Sushil K. Gupta, Vestal, NY (US)

Correspondence Address: Mark Levy;SALZMAN & LEVY, 19 Chenango Street, Binghamton, NY, 13901, US

(21) Appl. No.: 10/678,922

(22) Filed: Oct. 6, 2003

(51) Int. Cl. A61K 31/4745; A61K 31/337; A61F 2/00

(52) U.S. Cl. 424/423; 514/291; 514/449

(57) ABSTRACT

A suture, graft, or suture/graft combination that functions as a drug delivery system. In one embodiment of the invention, a suture fabricated from a polymer material incorporates the therapeutic agent within its pores, and then osmotically delivers the therapeutic agent directly to the injury. In a second embodiment of the invention, the suture is coated with an active ingredient that is time released (leaches from the coating) into the sutured area.

### FIELD OF THE INVENTION

The present invention relates to drug delivery systems and, more particularly, to a suture, graft, or graft/suture combination that can deliver biologically active ingredients in order to inhibit neointimal tissue proliferation in the prevention of restenosis.

### BACKGROUND OF THE INVENTION

In the art of grafting, fifty-three percent of native vein grafts tend to narrow (restenosis) over a period of about one month to two years.

Pharmacologic inhibition of smooth muscle proliferation has been successful in animals, but not in humans. Prosthetic grafts placed into the arterial circulation in humans do not develop a complete endothelial lining. Endothelial cells from the adjacent native vessel migrate only short distances (usually less than 1 cm) into the proximal and distal ends of the prosthetic graft. The remaining graft lumen becomes lined with a “pseudointima,” which is composed of fibrin, collagen, and several absorbed proteins. Prosthetic graft failure is not always the result of primary thrombosis and stenotic lesions related to intimal hyperplasia. The failure can develop in the native vessel adjacent to an anastomosis.

Similar problems are noted in all vascular anastomosis. Intimal hyperplasia has been reported to be a significant cause of failure for bypass grafts.

Several recent experiments for preventing smooth muscle cell (SMC) proliferation have shown promise, although the mechanisms are unclear. Heparin is the most commonly used agent for causing inhibition of SMC proliferation, both in vitro, and in animal models having a balloon angioplasty, mediated injury. The mechanism of SMC inhibition using heparin is not known, but this may be due to any or all of the following: reduced expression of the growth regulatory protooncogens c-fos and c-myc, reduced cellular production of tissue plasminogen activator, and binding and sequestration of growth regulatory factors such as fibrovalent growth factor (FGF).

Other agents that have demonstrated the ability to reduce myointimal thickening in animal models of balloon vascular injury are: angiopeptin (a somatostatin analog), calcium channel blockers, angiotensin converting enzyme inhibitors (captopril, cilazapril), cyclosporin A, trapidil (an antianginal, antiplatelet agent), terbinafine (antifungal), colchicines and taxol (antitubulin antiproliferatives), and c-myc and c-myb antisense oligonucleotides.

Additionally, a goat antibody to the SMC amidogen platelet derived growth factor (PDGF) has been shown to be effective in reducing myointimal thickening in a rat model of balloon angioplasty injury, thereby implicating PDGF directly in the etiology of restenosis. Thus, while no therapy has as yet proven clinically successful in preventing restenosis after angioplasty, the in vivo experimental success of several agents known to inhibit SMC growth suggests that as a class these agents have the capacity to prevent clinical restenosis, thus deserving careful evaluation in humans.

Over 200,000 vascular conduits are implanted by vascular surgeons each year. Intimal hyperplasia has been a cause of failure of a significant number of these conduits, thus causing recurrent surgeries and the loss of limbs.

In the western world, coronary heart disease is the major cause of death in men over the age of forty and in women over the age of fifty. Most coronary artery-related deaths are due to atherosclerosis. Atherosclerotic lesions, which limit or obstruct coronary blood flow, are the major cause of ischemic heart disease-related mortality.

Atherosclerosis results in 500,000 to 600,000 deaths in the United States annually. To arrest the disease process and to prevent the more advanced disease states in which the cardiac muscle itself is compromised, direct intervention has been employed via percutaneous transluminal coronary angioplasty (PTCA) or coronary artery bypass graft (CABG).

In the normal arterial wall, smooth muscle cells (SMC) proliferate at a low rate (0.1%/day) in vessel walls having a contractile phenotype that is characterized by 80% to 90% of the cell cytoplasmic volume supporting the contractile apparatus. Endoplasmic reticulum, golgi bodies, and free ribosomes are rarely found, and when located appear in the perinuclear region. Extracellular matrix surrounds SMC and is rich in heparin-like glycosylaminoglycans, which are believed to be responsible for maintaining SMC in the contractile phenotypic state.

Surgical intervention of the vessels will cause injury to the smooth muscle cells within the arterial wall. Cell derived growth factors such as platelet-derived growth factor (PDGF), basic fibroblast growth factor (BFGF), and epidermal growth factor (EGF) release form platelets (i.e., PDGF) adhering to the damaged arterial luminal surface. They invade macrophages and/or leukocytes, or form directly within the SMC (i.e., BFGF). They provoke a proliferation and migratory response in medial SMC. These cells undergo a phenotypical change from the contractile phenotype to a synthetic phenotype characterized by only a few contractile filament bundles. The cells comprise extensive rough endoplasmic reticulum, golgi, and free ribosomes. Proliferation/migration usually begins within one to two days after injury, and peaks at two days in the media, rapidly declining afterwards.

SMC proliferation, migration, and restenosis can be pharmacologically treated and prevented, because smooth muscle proliferation and migration are intimately involved with the pathophysiological response to arterial injury.

The present invention seeks to deliver such pharmacological treatment directly from the suture, the graft, or a suture/graft combination.

### DISCUSSION OF RELATED ART

At present, the art of suturing is focused on fabricating sutures that are inert, rather than providing suture materials that are pharmacologically active. To the best of current knowledge and belief, no attempt has been made to modify sutures, grafts, or suture/graft combinations to function as drug delivery systems to prevent SMC proliferation in the cell walls of the area of vessel injury.

In United States Patent Publication No. US20020055759, a bioactive surgical suture is illustrated. The surgical sutures are coated with biological agents for fighting cancer.

In United States Patent Publication No. US20010036948, a method and composition for inhibiting smooth muscle cell proliferation at a vascular injury site is shown.

In U.S. Pat. No. 5,733,925, issued to Kunz et al. on Mar. 31, 1998 for THERAPEUTIC INHIBITOR OF VASCULAR SMOOTH MUSCLE CELLS, a method for inhibiting stenosis following vascular trauma or disease is illustrated.

### SUMMARY OF THE INVENTION

In accordance with the present invention, a therapeutic agent is delivered to the site of arterial or vessel injury by a suture, graft, or suture/graft combination. In one embodiment of the invention, a suture fabricated from a polymer material such as polypropylene or PTFE, contains the therapeutic agent within its reservoir, and then osmotically delivers the agent through its pores to the injury. In a second embodiment of the invention, the suture is coated with an active ingredient that is time released into the sutured area. Such an active ingredient, e.g., taxol, rapamycin, and derivates thereof, inhibits the smooth muscle cell migration (SMC).

The ideal coating material is designed to strongly adhere to the original suture material, and the coating retains the drug at a sufficient load level to deliver the required dose. Plasma-assisted coatings have been found useful for this purpose. The release of the drug is designed in a controlled way, delivering the active agent over a period of several weeks. The coating thickness is made as thin as possible to minimize the wall profile.

In addition, the coating material is inert in the sense that it cannot invoke any adverse response by the body, i.e., it is non-thrombogenic, non inflammatory, etc.

An alternative embodiment uses synthetic graft material coated or incorporated with the biologically active ingredient to reduce smooth muscle cell proliferation.

It is an object of this invention to provide a suture, graft, or suture/graft combination that functions as a drug delivery system.

It is another object of the present invention to provide a suture, graft, or suture/graft combination that prevents or reduces smooth muscle cell proliferation (SMC) in the cell walls of the area of vessel injury.

It is a further object of this invention to provide a suture, graft, or suture/graft combination that delivers a biological agent without causing any adverse body reactions.

### DESCRIPTION OF THE PREFERRED EMBODIMENT

Generally speaking, the invention features a suture, graft, or suture/graft combination that functions as a drug delivery system. In one embodiment of the invention, a suture fabricated from a polymer material incorporates the therapeutic agent within its pores and then osmotically delivers the therapeutic agent directly to the injury. In a second embodiment of the invention, the suture is coated with an active ingredient that is time released (leaches from the coating) into the sutured area.

Pharmacological attempts to prevent restenosis by pharmacologic means have thus far been unsuccessful. All of these unsuccessful attempts comprise systemic administration of the trial agents. Aspirin-dipyridamole, ticlopidine, acute heparin administration, chronic warfarin (six months administration), and methylprednisolone have been ineffective in preventing restenosis.

Platelet inhibitors, however, have been effective in preventing acute reocclusion after angioplasty. The calcium antagonists have also been unsuccessful in preventing restenosis, although they are still under study. Other agents currently being studied include thromboxane inhibitors, prostacyclin mimetics, platelet membrane receptor blockers, thrombin inhibitors, and angiotensin converting enzyme inhibitors. These agents must be given systemically, however, and attainment of a therapeutically effective dose may not be possible. Antiproliferative (or anti-restenosis) concentrations may exceed the known allowable toxic concentrations of these agents so that levels sufficient to produce smooth muscle inhibition may not be reached.

Other clinical trials for preventing restenosis have involved the use of dietary fish oil supplements, thromboxane receptor antagonists, cholesterol lowering agents, and serotonin antagonists. These trials have given either conflicting or negative results. Thus, many promising agents lack the delivery system to prevent intimal hyperplasia.

There are reports of stents that appear useful in preventing or reducing the proliferation of restenosis. In trials using heparin-coated stents, however, it appeared that they possess the same benefit of reduction in stenosis diameter as was observed with non-heparin coated stents. Heparin coating appears to have the added benefit of reducing sub-acute thrombosis after stent implantation, leading to the conclusion that sustained mechanical expansion of a stenosed coronary artery provides some measure of restenosis prevention, and coating of stents with heparin-like agents appears to be feasible.

Numerous agents are being actively studied as antiproliferative agents for use in restenosis and have shown some activity in experimental animal models. These include: heparin and heparin fragments, taxol, angiotensin converting enzyme (ACE) inhibitors, cyclosporin A, steroids, ionizing radiation, fusion toxins, antisense oligonucleotides, gene vectors, and rapamycin.

Rapamycin is of particular interest for use with sutures and grafts in this invention. Rapamycin is a macrolide antibiotic, which blocks IL-2-mediated “T” cell proliferation, and possesses anti-inflammatory activity. The precise mechanism by which rapamycin accomplishes its result is still under active investigation.

Rapamycin has been shown to prevent the G.sub.I to S phase progression of T-cells through the cell cycle by inhibiting specific cell cyclins and cyclin-dependent protein kinases. The antiproliferative action of rapamycin is not limited to T-cells. It has been demonstrated that rapamycin prevents proliferation of both rat and human SMC in vitro, and the rat, porcine, and human SMC migration can also be inhibited by rapamycin. Thus, rapamycin is capable of inhibiting both the inflammatory response known to occur after arterial injury and stent implantation, as well as the SMC hyperproliferative response. Thus, a suture or graft with rapamycin delivery would be most useful for the purposes of this invention.

Another useful drug in this invention is taxol (Paclitaxel, Taxane, and its derivatives including, but not limited to, natural and synthetic, water-soluble, and non water-soluble substances). Taxol and its derivatives may be used in this invention as a natural product, or in its synthetic form. The natural product is extracted from Taxus Chinensis without any semi-synthesis process. Paclitaxel is a white to off-white crystalline powder, and is highly lipophilic (insoluble in water). It can be mixed with a polymer carrier solution that is coated upon the suture or graft to make it more soluble and readily available at local sites.

In the treatment of restenosis or hyperplasia prevention, it is important to provide objectives for the suture or graft, viz., it should inhibit local thrombosis without the risk of systemic bleeding complications, and it should provide continuous prevention of the squeal of arterial injury, including local inflammation and sustained prevention of smooth muscle proliferation at the site of angioplasty. All of this must be accomplished without serious systemic complications. Agents that reduce or prevent inflammation and the proliferation of SMC, when combined with a suture, graft, or suture/graft combinations, are most efficacious in the prevention of graft failure from intimal hyperplasia.

The invention uses plasma-assisted coatings to provide delivery of the aforementioned drugs. These types of coatings provide complex delivery strategies. Their materials and their techniques for coating sutures and grafts do not pose any harm to the drugs. The surface energies and surface chemical activity can be altered without affecting bulk properties. Depositing polymers by this method allows the thickness to be accurately controlled so that the profile is minimized on the suture or graft wall.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A suture, graft or suture/graft combination comprising PTFE and having a polymer coating containing releasable biological agents for the prevention and reduction of intimal hyperplasia, smooth muscle cell proliferation in a sutured, grafted, or injured area of a vessel or arterial wall.

2. The suture, graft, or suture/graft combination in accordance with claim 1, wherein said polymer coating is a plasma-assisted coating deposited upon the suture, graft, or suture/graft combination.

3. The suture, graft, or suture/graft combination in accordance with claim 1, wherein said biological agents are selected from a group of substances consisting of: rapamycin, taxol, derivatives of taxol and rapamycin, and combinations thereof.

4. A suture, graft or suture/graft combination comprising PTFE having a reservoir containing releasable biological agents for the prevention and reduction of intimal hyperplasia, smooth muscle cell proliferation in a sutured, grafted area or injured area of a vessel or arterial wall, said biological agent being osmotically released through the pores in the reservoir.

5. The suture, graft, or suture/graft combination in accordance with claim 4, wherein said biological agents are selected from a group of substances consisting of: rapamycin, taxol, derivatives of taxol and rapamycin, and combinations thereof.

6. The suture, graft, or suture/graft combination in accordance with claim 4, wherein said biological agents have means for timed release from the suture, graft, or suture/graft reservoir.
